(19) United States
(12) Patent Application Publication
(10) Pub. No.: US 2007/0142849 A1
(43) Pub. Date: Jun. 21, 2007

(54) Helical tissue manipulation instruments and methods of use

(75) Inventors: Richard C. Ewers, Fullerton, CA (US); Arvin T. Chang, West Covina, CA (US); John A. Cox, Rancho Santa Margarita, CA (US)

Correspondence Address: LEVINE BAGADE HAN LLP, 2483 EAST BAYSHORE ROAD, SUITE 100, PALO ALTO, CA, 94303, US

(73) Assignee: USGI Medical, Inc., San Clemente, CA (US)

(21) Appl. No.: 11/303,521

(22) Filed: Dec. 16, 2005

(51) Int. Cl. A61B 17/08

(52) U.S. Cl. 606/153

(57) ABSTRACT

Helical tissue manipulation instruments and methods of their use are described herein. A helical tissue engager is adapted to reversibly engage tissue and is positioned upon a flexible shaft which is advanceable through a rigidizable endoscopic assembly. The flexible shaft defines a marked section proximal to the tissue engager which can include any number of markings, designs, patterns, projections, textures, etc., which acts to provide a visual indication to the user as to the translational movement, rotation, direction of rotation, etc., of the tissue engager and the shaft. An optional guidewire can be advanced through the tissue engager. Additionally, the tissue engager and shaft can be advanced through an optional tubular sheath which may be used for dilating tissue openings prior to passage of the helical engager through the tissue opening.

### BACKGROUND OF THE INVENTION

### Field of the Invention

The present invention relates to methods and apparatus for manipulating and/or securing tissue. More particularly, the present invention relates to methods and instruments for manipulating and/or securing tissue endoluminally, for instance, to form and/or secure tissue folds or to approximate regions of tissue, etc.

A number of surgical techniques have been developed to treat various gastrointestinal disorders. Many of the surgical procedures require regions of tissue within the body to be engaged, manipulated, and/or reliably secured. The gastrointestinal lumen, for instance, includes four tissue layers, where the mucosa layer is the inner-most tissue layer followed by connective tissue, the muscularis layer, and where the serosa layer is the outer-most tissue layer.

One problem with conventional endoluminal or laparoscopic tissue engagement instruments is the ability of the instruments to reliably engage at least the muscularis tissue layer in order to provide for secure tissue manipulation and securement. One method for temporarily engaging tissue within a body lumen is to utilize graspers to hold and manipulate the tissue to suture the stomach wall into folds. However, many graspers provide for inadequate purchase of the tissue, particularly when the tissue is stretched or the instrument is angled relative to the tissue surface.

Another method for temporary tissue engagement utilizes vacuum engagement for adhering the tissue to the instrument. However, such methods may interfere with maintaining the insufflation of internal body lumens. Yet another method involves utilizing a helical coil positioned upon a flexible shaft for engaging tissue to the helical coil by torquing the shaft and coil into the tissue.

An example of such an endoluminal tissue engager is shown and described in U.S. Pat. Pub. 2004/0193117 A1 (Laufer et al.). However, because such instruments are rotated about their longitudinal shafts for engaging the tissue, it is difficult to determine the depth to which the helical coil has been advanced into the tissue. Moreover, when such tools are over-torqued torqued relative to the tissue, they have a tendency to wrap or wad a portion of the tissue not only about the helical coil but also about the shaft as well.

Accordingly, there exists a need for a tissue engagement instrument which overcomes the problems described above.

### BRIEF SUMMARY OF THE INVENTION

In engaging, manipulating, and/or securing the tissue, various methods and devices may be implemented. When manipulating and securing tissue within a patient's body, an elongate shaft having a helical tissue engager on or near the distal end of the shaft may be utilized in conjunction with a tissue manipulation assembly. Such an instrument may be generally utilized in endoluminal procedures where the tools are delivered through an endoscopic device.

In one example, a helical tissue engagement member may be delivered through a rigidizable endoscopic assembly, for instance as described in detail in U.S. patent application Ser. No. 10/734,562 filed Dec. 12, 2003 and in U.S. patent application Ser. No. 10/346,709 filed Jan. 15, 2003, both of which are incorporated herein by reference in its entirety. The helical tissue engagement member may be configured as a tissue piercing helix or corkscrew structure upon flexible shaft which may be rotated about its longitudinal axis to engage the tissue of interest by rotating its handle located on the proximal end of the flexible shaft.

The helical tissue engagement member and the flexible shaft are rotated about its longitudinal axis to advance the engagement member into the tissue region of interest. A distal portion of the shaft proximal to the engagement member (or the entire length or a majority of the length of shaft in other variations) may include a marked section. Accordingly, the marked section may comprise any number of markings, designs, patterns, projections, textures, etc., which acts to provide a visual indication to the user as to the translational movement, rotation, direction of rotation, etc., of the engagement member and the shaft relative to the tissue region when viewed from outside the patient body laparoscopically or endoluminally, for instance, through a visual lumen.

In one variation, the visual indicators may be patterned as longitudinal stripes along the length of the shaft. The longitudinal stripes may be positioned around the outer surface of the shaft with uniform or irregular spacing relative to one another. The longitudinal stripes may serve to visually indicate to the user the direction of rotation of the engagement member and the shaft relative to the tissue surface, particularly when viewed through the visual lumen. The longitudinal stripes may also serve to indicate when the shaft is torqued or over-torqued against the tissue. The flexible shaft may be made in various colors, e.g., silver, grey, black, blue, etc., while the stripes in the marked section may also be made in various colors to contrast against the color of the shaft.

An optionally removable handle may be positioned upon a proximal end of the flexible shaft and may be configured in alternative configurations depending upon the desired ergonomics. Moreover, the flexible shaft may be alternatively configured into a rigid shaft, straightened or angled, for laparoscopic applications.

Another variation of the tissue engagement instrument may be connectable via a cable to a power supply located externally of the patient body. The power supply may be set to any number of energy modalities, for instance, radio-frequency, microwave, thermal, etc., for supplying energy to the helical tissue engagement member either at its tip or along its length for any number of procedures. For instance, the tip of the engagement member may be energized to facilitate cutting or piercing of the member into tissue. Alternatively, the length of the engagement member may be energized to provide for coagulation of the contacted tissue or the surrounding tissue, depending upon the type of transmitted energy. The cable may be optionally removable from the handle and the use of power supply may be omitted altogether during a procedure.

At least one or more longitudinal stripes may be patterned over the outer surface of the shaft to provide the visual indicator for shaft motion. As described above, the longitudinal stripes may be positioned around the outer surface of the shaft with uniform or irregular spacing relative to one another. An additional band may also be provided upon the distal tip of the shaft proximal to the tissue engagement member and distal to the longitudinal stripes. The band may be of a known length, e.g., 1 mm to 1 cm or greater, to provide a visual indication of the depth that the engagement member or the shaft has been advanced into the tissue surface.

Another variation of the marked section may be in a spiral pattern, as typically seen on conventional guidewires, to indicate translational and rotational movement of the flexible shaft. Yet another variation may include a flexible shaft with a circumferential stop positioned at a distal end of the shaft, which may have a diameter which is larger than a diameter of the shaft and may be utilized to act as a stop to prevent the engagement member from being advanced beyond a predetermined location.

Yet another variation may include a helical engagement member having an elongated length extending from the engagement member to the distal end of the flexible shaft. The elongated length may extend anywhere from 1 mm to several millimeters or longer and may function to prevent engaged tissue from becoming wedged or pinched between the engagement member and the end of the shaft.

Additionally, the helical engagement member may also be utilized with a guidewire passed through a lumen defined through the flexible shaft and the helical engagement member for various procedures. Moreover, the engagement member, with or without the guidewire, may also be passed through a dilating sheath member having an opening defined along its length for passing the engagement member therethrough.

### BRIEF DESCRIPTION OF THE DRAWINGS

FIG. 1 illustrates one example in which a rigidizable endoscopic assembly may be advanced into a patient's stomach per-orally and through the esophagus with a tissue manipulation assembly advanced through a first lumen and a helical tissue engagement instrument advanced through a second lumen.

FIGS. 2A to 2C illustrate an example for performing an endoluminal tissue manipulation and securement procedure utilizing a tissue manipulation assembly in combination with a helical tissue engagement instrument within, e.g., a patient's stomach.

FIGS. 3A and 3B show close-up side and perspective detail views, respectively, of an engagement member and flexible shaft exiting a lumen of the rigidizable endoscopic assembly.

FIGS. 4A and 4B show side and perspective views, respectively, of a variation of the helical tissue engagement instrument having a handle positioned upon its proximal end.

FIG. 4C shows an assembly view of another variation illustrating an optional power supply connectable via a cable to the tissue engagement instrument.

FIG. 5 shows yet another variation where the handle may be removable from the flexible shaft of the tissue engagement instrument.

FIGS. 6A and 6B show side and perspective detail views, respectively, of another variation of the marked section of the flexible shaft having at least one or more longitudinal stripes patterned over the outer surface of the shaft.

FIG. 7 shows yet another variation of the marked section having an additional band provided upon the distal tip of the shaft proximal to the tissue engagement member and distal to the longitudinal stripes.

FIG. 8 shows yet another variation having a spiral marked section.

FIG. 9 shows yet another variation of a flexible shaft including a circumferential stop positioned at a distal end of the shaft.

FIG. 10 shows another variation of the tissue engagement instrument where the helical engagement member includes an elongated length extending from the engagement member to the distal end of the flexible shaft.

FIGS. 11A and 11B illustrate one method for utilizing the longitudinal stripes, in particular, to indicate a state of over-torquing the shaft against tissue.

FIG. 12 illustrates another variation of the tissue engagement instrument defining a hollow lumen which is sized appropriately for a guidewire to be passed therethrough and through the center of the helical tissue engagement member.

FIGS. 13A and 13B show perspective views of a dilating sheath, optionally having visual markings, which may be utilized with the tissue engagement instrument for facilitating transgastric entry of the flexible shaft through a gastrotomy from within the stomach and into the peritoneal cavity.

FIG. 13C shows another variation of a dilating sheath having an energizable element or wire disposed upon the tip portion for facilitating the cutting or widening of a tissue opening.

FIGS. 14A to 14C illustrate perspective views of one example for using a tissue engagement instrument and a guidewire with the optional dilating sheath.

FIGS. 15A and 15B show detail perspective views of an example showing the optional sheath positioned within a lumen of the rigidizable endoscopic assembly.

FIG. 16 illustrates another variation of the sheath and flexible shaft positioned through an opening of the rigidizable endoscopic assembly having a tapered atraumatic tip which is eccentrically angled such that the opening of the lumen, where the sheath and flexible shaft exit, defines the distal apex of the tapered tip.

### DETAILED DESCRIPTION OF THE INVENTION

In manipulating tissue or creating tissue folds, a distal end effector may be advanced endoluminally, e.g., transorally, transgastrically, percutaneously, etc., into the patient's body, e.g., the stomach. The tissue may be temporarily engaged or grasped and the engaged tissue may be manipulated by a surgeon or practitioner from outside the patient's body. Examples of creating and forming tissue plications may be seen in further detail in U.S. patent application Ser. No. 10/955,245 filed Sep. 29, 2004 as well as in U.S. patent application Ser. No. 10/735,030 filed Dec. 12, 2003, each of which is incorporated herein by reference in its entirety.

In engaging, manipulating, and/or securing the tissue, various methods and devices may be implemented. For instance, tissue securement devices may be delivered and positioned via an endoscopic apparatus for contacting a tissue wall of the gastrointestinal lumen, creating one or more tissue folds, and deploying one or more tissue anchors through the tissue fold(s). The tissue anchor(s) may be disposed through the muscularis and/or serosa layers of the gastrointestinal lumen. When manipulating and securing tissue within a patient's body, a separate elongate shaft having a helical tissue engager on or near the distal end of the shaft may be utilized in conjunction with a tissue manipulation assembly. Such an instrument may be generally utilized in endoluminal procedures where the tools are delivered through an endoscopic device.

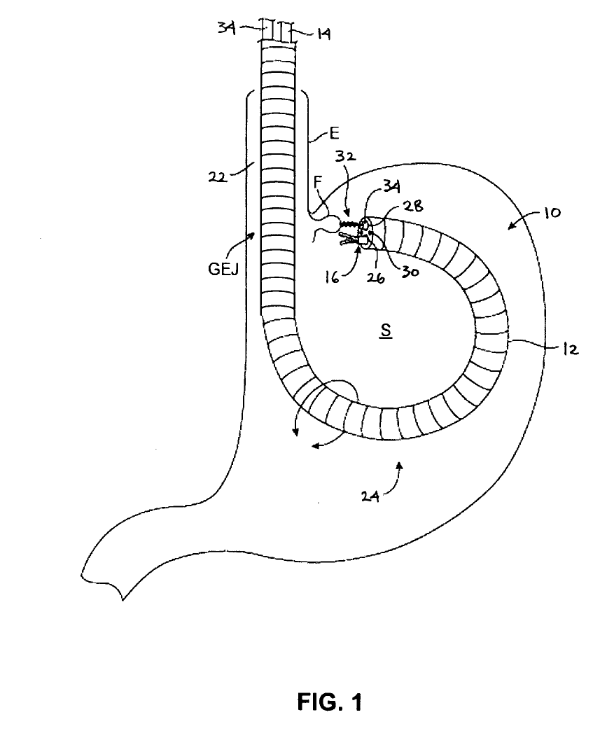

As illustrated in FIG. 1, one such example is shown in which a shape-lockable endoscopic assembly 10 may be advanced into a patient's stomach S per-orally and through the esophagus E. Such an endoscopic assembly 10 may generally comprise an endoscopic device which may have a distal portion which may be articulated and steered to position its distal end anywhere within the stomach S. Once desirably configured, assembly 10 may then be locked or rigidized to maintain its shape or configuration to allow for procedures to be performed on the tissue utilizing any number of tools delivered through the assembly 10. Shape-lockable assembly 10 and its variations are described in further detail in U.S. patent application Ser. No. 10/734,562 filed Dec. 12, 2003 and in U.S. patent application Ser. No. 10/346,709 filed Jan. 15, 2003, both of which are incorporated herein by reference in its entirety.

Shape-lockable assembly 10 may be generally comprised of shape-lockable endoscopic body 12 having an articulatable distal portion 24. The endoscopic body 12 may define at least first and second lumens 26, 28, respectively, through the endoscopic body 12 through which one or more tools may be deployed into the stomach S. Additional lumens may be provided through shape-lockable endoscopic body 12, such as a visualization lumen 30, through which an endoscope may be positioned to provide visualization of the region of tissue. Alternatively, an imager such as a CCD imager or optical fibers may be provided in lumen 30 to provide visualization. An optional thin wall sheath may be disposed through the patient's mouth, esophagus E, and possibly past the gastroesophageal junction GEJ into the stomach S. Shape-lockable body 12, having a covering 22 thereon, may be advanced through esophagus E and into stomach S while disposed in a flexible state.

Distal steerable portion 24 of endoscopic body 12 may be then articulated to an orientation, e.g., whereby distal portion 24 facilitates engagement of tissue near and/or inferior to the patient's gastroesophageal junction GEJ. Accordingly, distal steerable portion 24 may comprise a number of steering features, as described in further detail in U.S. patent application Ser. Nos. 10/734,562 and 10/346,709, incorporated above. With distal steerable portion 24 disposed in a desired configuration or orientation, endoscopic body 12 may be reversibly shape-locked to a rigid state such that the endoscopic body 12 maintains its position within the stomach S. Various methods and apparatus for rigidizing endoscopic body 12 along its length are also described in further detail in U.S. patent application Ser. Nos. 10/734,562 and 10/346,709, incorporated above.

FIG. 1 further shows tissue manipulation assembly 16 having been advanced through first lumen 26 and a helical tissue engagement member 32 positioned upon flexible shaft 34 advanced through second lumen 28. As the tissue wall of a body lumen, such as the stomach, typically comprises an inner mucosal layer, connective tissue, the muscularis layer and the serosa layer. To obtain a durable purchase, e.g., in performing a stomach reduction procedure, helical tissue engagement member 32 may be advanced into contact with the tissue and preferably engages the tissue F such that when the tissue engagement member 32 is pulled proximally to draw the engaged tissue F between the jaw members 18, 20 of tissue manipulation assembly 16, at least the muscularis tissue layer and the serosa layer is drawn into tissue manipulation assembly 16. As tissue manipulation assembly 16 may be utilized to grasp and secure the engaged tissue, any number of tools may be utilized with tissue manipulation assembly 16, e.g., through shape-lockable endoscopic body 12, to engage and manipulate the tissue of interest relative to tissue manipulation assembly 16.

An illustrative example of a tissue manipulation instrument which may be utilized for endoluminally accessing tissue is described in further detail in U.S. patent application Ser. No. 11/070,863 filed Mar. 1, 2005 (U.S. Pat. Pub. 2005/0251166 A1), which is incorporated herein by reference in its entirety. Such an instrument assembly generally comprises a flexible catheter or tubular body 14 which may be configured to be sufficiently flexible for advancement into a body lumen, e.g., transorally, percutaneously, laparoscopically, etc. Tubular body 14 may be configured to be torqueable through various methods, e.g., utilizing a braided tubular construction, such that when a proximally-located handle is manipulated and/or rotated by a practitioner from outside the patient's body, the longitudinal and/or torquing force is transmitted along body 14 such that the distal end of body 14 is advanced, withdrawn, or rotated in a corresponding manner.

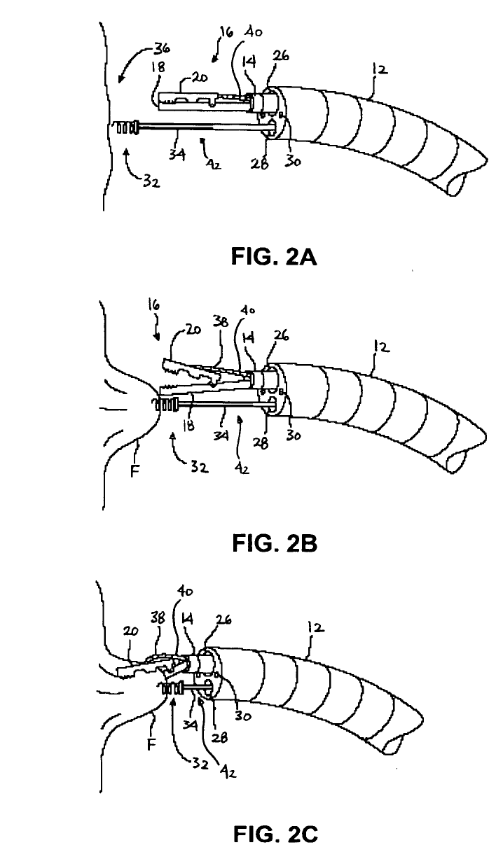

As shown in FIGS. 2A to 2C, tissue manipulation assembly 16 is located at the distal end of tubular body 14 and is generally used to contact and form tissue folds, as mentioned above. The tissue manipulation assembly 16 may be connected to the distal end of tubular body 14 via a pivotable coupling. Lower jaw member 18 extends distally from the pivotable coupling and upper jaw member 20, in this example, may be pivotably coupled to lower jaw member 18 via a jaw pivot. The location of the jaw pivot may be positioned at various locations along lower jaw 18 depending upon a number of factors, e.g., the desired size of the “bite” or opening for accepting tissue between the jaw members, the amount of closing force between the jaw members, etc. One or both jaw members 18, 20 may also have a number of protrusions, projections, grasping teeth, textured surfaces, etc., on the surface or surfaces of the jaw members 18, 20 facing one another to facilitate the adherence of tissue between the jaw members 18, 20.

Launch tube 40 may extend from the handle, through tubular body 14, and distally from the end of tubular body 14 where a distal end of launch tube 40 is pivotally connected to upper jaw member 20 at a launch tube pivot. A distal portion of launch tube 40 may be pivoted into position within a channel or groove defined in upper jaw member 20, to facilitate a low-profile configuration of tissue manipulation assembly 16. When articulated, either via launch tube 40 or other mechanism, as described further below, jaw members 18, 20 may be urged into an open configuration to receive tissue in the jaw opening between the jaw members 18, 20.

Launch tube 40 may be advanced from its proximal end at the handle such that the portion of launch tube 38, which extends distally from body 14, is forced to rotate at a hinge or pivot and reconfigure itself such that the exposed portion forms a curved or arcuate shape that positions the launch tube opening perpendicularly relative to upper jaw member 20. Launch tube 40, or at least the exposed portion of launch tube 38, may be fabricated from a highly flexible material or it may be fabricated, e.g., from Nitinol tubing material which is adapted to flex, e.g., via circumferential slots, to permit bending.

FIGS. 2A to 2C further illustrate one method for articulating a tissue manipulation assembly into an opened and closed configuration. As shown in FIG. 2A, the assembly may be delivered into a patient while in a low-profile configuration 40, e.g., trans-orally, trans-anally, percutaneously, through an endoscope, an endoscopic device, directly, etc., and desirably positioned relative to a tissue region of interest 36. The endoscopic body 12 may be rigidized to maintain its configuration within the patient body. Alternatively, it may be left in a flexible state during the procedure.

The tissue region of interest 36 as well as the procedure may be visualized through visualization lumen 30 or a separate imager. In either case, tissue manipulation assembly 16 and helical tissue engagement member 32 may be advanced distally out from endoscopic body 12 through their respective lumens 26, 28. Tissue engagement member 32 may be advanced into contact against the tissue surface, as shown in FIG. 2A, and then rotated via its proximal handle until the tissue is engaged. The engaged tissue F may be pulled proximally relative to endoscopic body 12 and tissue manipulation assembly 16 may be actuated via its proximally located handle into an open expanded jaw configuration for receiving the engaged tissue F, as shown in FIG. 2B.

Once desirably positioned, launch tube 40 may be urged proximally via its proximal end at the handle. Because of the jaw assembly pivot and the relative positioning of the upper jaw 20 along lower jaw member 18 and the launch tube pivot along upper jaw member 20, the proximal movement of launch tube 40 may effectively articulate upper jaw 20 into an expanded jaw configuration, as shown in FIG. 2B. Proximally urging launch tube 40 may also urge lower jaw member 18 to pivot and form an angle relative to a longitudinal axis of tubular body 14. The opening of upper jaw 20 relative to lower jaw 18 creates a jaw opening for grasping, receiving, and/or manipulating tissue. Moreover, the tissue manipulation assembly may also include a stop located adjacent to the jaw assembly pivot or within the pivot itself.

Once launch tube 40 has been urged proximally, it may be locked into place thus locking the jaw configuration as well. Moreover, having the launch tube 40 articulate the jaw members 18, 20 in this variation eliminates the need for a separate jaw articulation and/or locking mechanism. Once the tissue has been pulled or manipulated between jaw members 18, 20, launch tube 40 may be pushed distally to actuate the jaw members 18, 20 into a closed, grasping configuration, as shown in FIG. 2C, for engagement with the tissue. As launch tube 40 is urged distally through elongate body 12, lower jaw member 18 may be maintained at an angle relative to the tissue to further facilitate manipulation of the grasped tissue.

Although launch tube 40 may be fabricated from different materials having differing flexibilities, it may also be fabricated from a single material, as mentioned above, where the flexible portion 38 may be configured, e.g., by slotting, to allow for bending of the launch tube 40 in a plane to form a single curved or arcuate section while the proximal rigid section may extend at least partially into tubular body 14 to provide column strength to launch tube 40 while it is urged distally upon upper jaw member 20 and upon any tissue engaged thereby, as seen in the FIG. 2C.

Once the tissue has been engaged between jaw members 18,20, a needle assembly may be urged through the handle and out through launch tube 40. The needle assembly may pass through lower jaw member 18 via a needle assembly opening defined in lower jaw member 18 to pierce through the grasped tissue. Once the needle assembly has been passed through the engaged tissue, one or more tissue anchors may be deployed for securing the tissue, as described in further detail in U.S. patent application Ser. No. 10/955,245, which has been incorporated by reference above.

Helical tissue engagement member 32 may be retracted from the tissue F or it may be left within the tissue while the tissue manipulation assembly engages and secures the tissue F. The helical tissue engagement member 32 is shown as a tissue piercing helix or corkscrew structure upon flexible shaft 34. Tissue engagement member 32 may be rotated about its longitudinal axis to engage the tissue of interest by rotating its handle located on the proximal end of flexible shaft 34.

A distal portion of shaft 34 proximal to engagement member 32 (or the entire length or a majority of the length of shaft 34 in other variations) may include a marked section 42, as shown in FIGS. 2A to 2C. Helical tissue engagement member 32 and flexible shaft 34 are rotated about its longitudinal axis to advance engagement member 32 into the tissue region of interest 36. Accordingly, marked section 42 may comprise any number of markings, designs, patterns, projections, textures, etc., which acts to provide a visual indication to the user as to the translational movement, rotation, direction of rotation, etc., of engagement member 32 and shaft 34 relative to tissue region 36 when viewed from outside the patient body laparoscopically or endoluminally, for instance, through visual lumen 30.

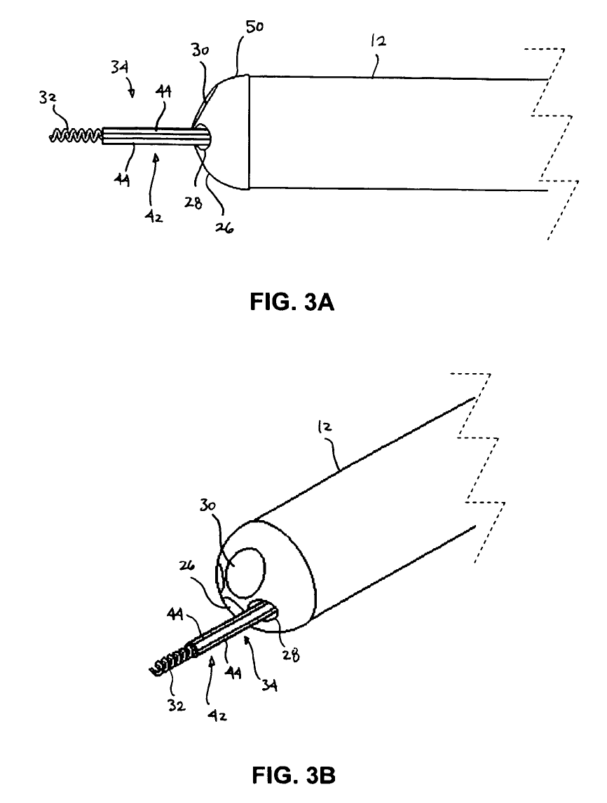

FIGS. 3A and 3B show close-up side and perspective detail views, respectively, of engagement member 32 and flexible shaft 34 exiting lumen 28 of body 12. Marked section 42 may be seen upon flexible shaft 34 proximal to engagement member 32. In this variation, the visual indicators are shown as longitudinal stripes 44 along the length of shaft 34. Longitudinal stripes 44 may be positioned around the outer surface of shaft 34 with uniform or irregular spacing relative to one another. Longitudinal stripes 44 may serve to visually indicate to the user the direction of rotation of engagement member 32 and shaft 34 relative to the tissue surface, particularly when viewed through lumen 30. The elongate body 12 is shown in this variation as having an atraumatic rounded distal tip 50. Other variations of marked section 42 are shown and described below in further detail.

Flexible shaft 34 may be made in various colors, e.g., silver, grey, black, blue, etc., while the stripes 44 in marked section 42 may also be made in various colors to contrast against the color of shaft 34. For example, if flexible shaft 34 were made in a blue color, stripes 44 may be made to have a black color to contrast against shaft 34. Other color variations and schemes may be devised, as desirable, and are intended to be within the scope of this disclosure.

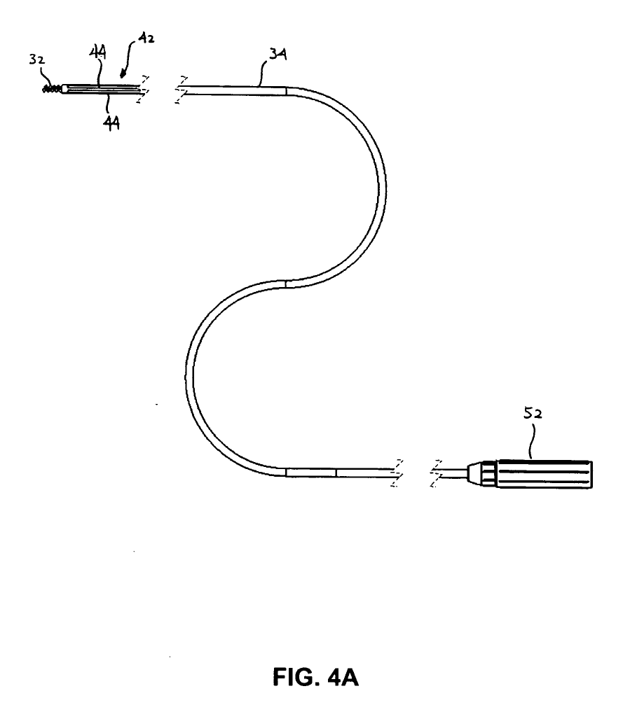

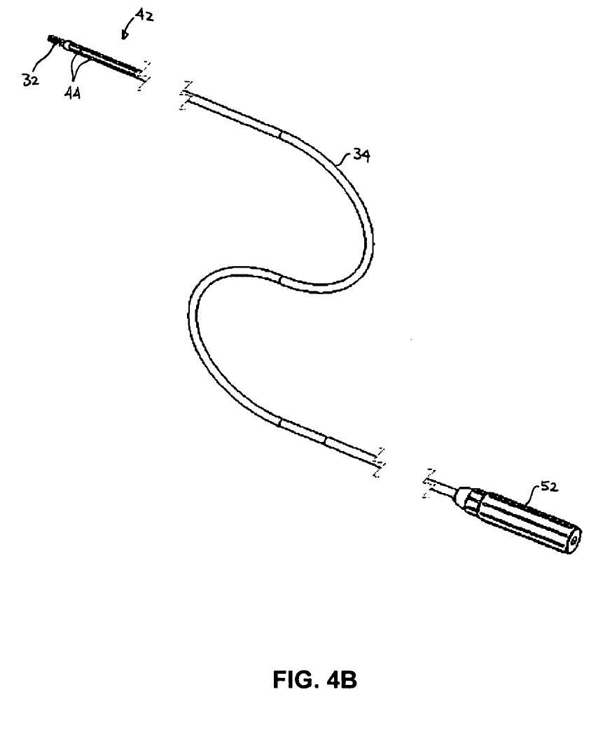

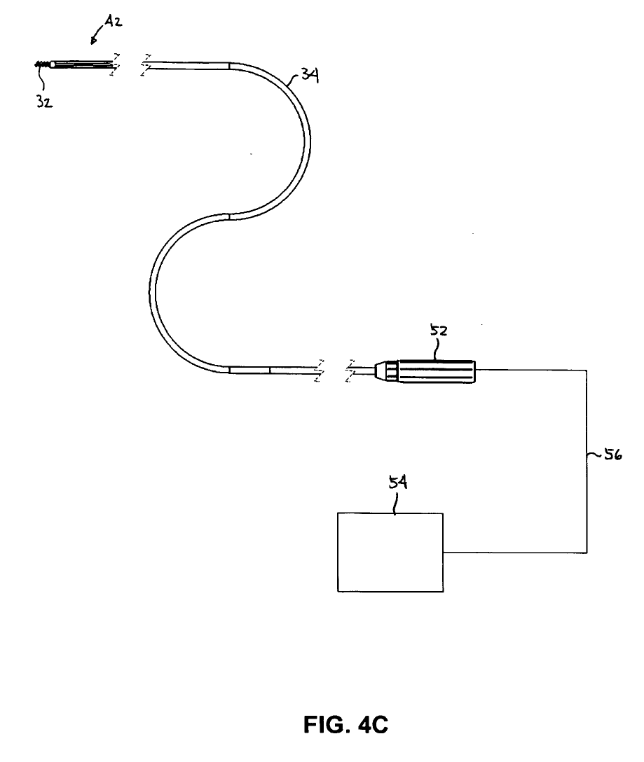

FIG. 4A shows a side view of one variation of the tissue engagement instrument having a handle 52 positioned upon a proximal end of flexible shaft 34. Handle 52 may be configured in alternative configurations depending upon the desired ergonomics. Moreover, flexible shaft 34 may be alternatively configured into a rigid shaft, straightened or angled, for laparoscopic applications. FIG. 4B shows a perspective view of the tissue engagement instrument having the marked section 42 defined on the outer surface of the distal portion of flexible shaft 34.

FIG. 4C shows yet another variation of the tissue engagement instrument connectable via a cable 56 to power supply 54, located externally of the patient body. Power supply 54 may be set to any number of energy modalities, for instance, radio-frequency, microwave, thermal, etc., for supplying energy to the helical tissue engagement member 32 either at its tip or along its length for any number of procedures. For instance, the tip of engagement member 32 may be energized to facilitate cutting or piercing of the member 32 into tissue. Alternatively, the length of engagement member 32 may be energized to provide for coagulation of the contacted tissue or the surrounding tissue, depending upon the type of transmitted energy. Cable 56 may be optionally removable from handle 52 and the use of power supply 54 may be omitted altogether during a procedure.

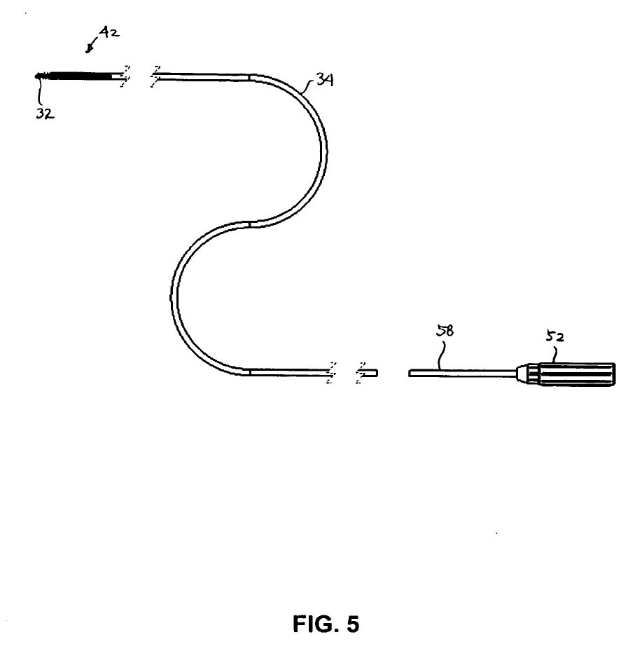

In yet another variation shown in FIG. 5, handle 52 may be attached to removable shaft 58, which may be removable from flexible shaft 34. This variation allows for the removal of handle 52 from the tissue engagement instrument. Such a variation may allow for the flexible shaft 34 and engagement member 32 to be back-loaded proximally through lumen 28 without handle 52.

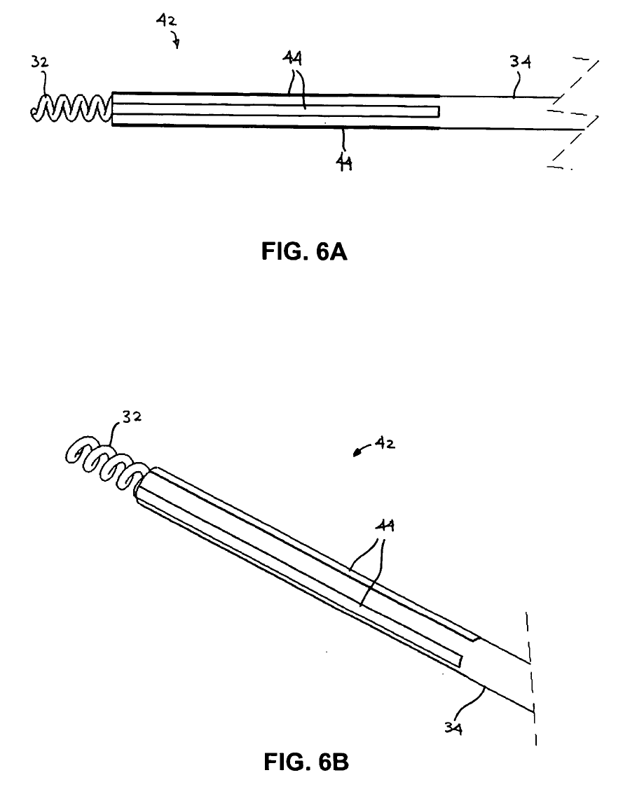

FIGS. 6A and 6B show side and perspective detail views of one variation of the marked section 42 of flexible shaft 34. As shown, at least one or more longitudinal stripes 44 may be patterned over the outer surface of shaft 34 to provide the visual indicator for shaft motion. As described above, longitudinal stripes 44 may be positioned around the outer surface of shaft 34 with uniform or irregular spacing relative to one another.

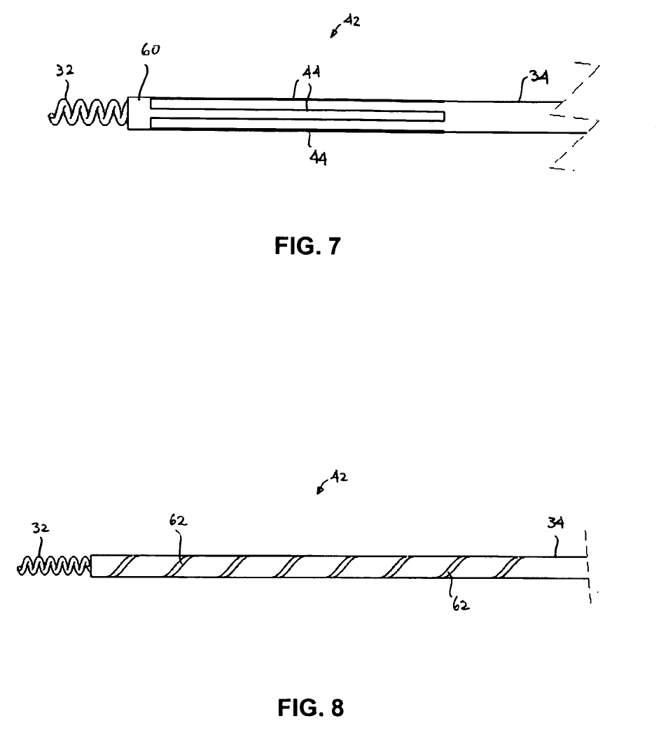

FIG. 7 shows yet another variation of marked section 42 where an additional band 60 may be provided upon the distal tip of shaft 34 proximal to tissue engagement member 32 and distal to longitudinal stripes 44. Band 60 may be of a known length, e.g., 1 mm to 1 cm or greater, to provide a visual indication of the depth that engagement member 32 or shaft 34 has been advanced into the tissue surface. Moreover, band 60 may be of a similar or dissimilar color relative to longitudinal stripes 44 and/or flexible shaft 34, depending upon the desired color scheme, as described above.

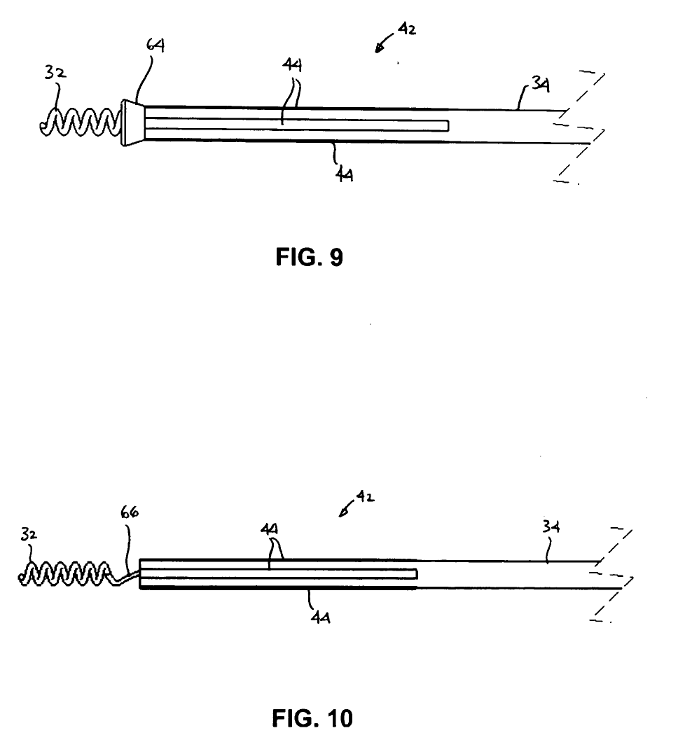

Another variation of marked section 42 is shown in the side view of FIG. 8 where the visual indicator may be in a spiral pattern 62, as typically seen on conventional guidewires, to indicate translational and rotational movement of flexible shaft 34. Yet another variation is shown in the side detail view of FIG. 9, which shows a flexible shaft 34 with a circumferential stop 64 positioned at a distal end of shaft 34. Stop 64 may have a diameter which is larger than a diameter of shaft 34 and may be utilized to act as a stop to prevent the engagement member 32 from being advanced beyond a predetermined location.

FIG. 10 shows another variation of the tissue engagement instrument where the helical engagement member 32 includes an elongated length 66 extending from engagement member 32 to the distal end of flexible shaft 34. Elongated length 66 may extend anywhere from 1 mm to several millimeters or longer and may function to prevent engaged tissue from becoming wedged or pinched between engagement member 32 and the end of shaft 34.

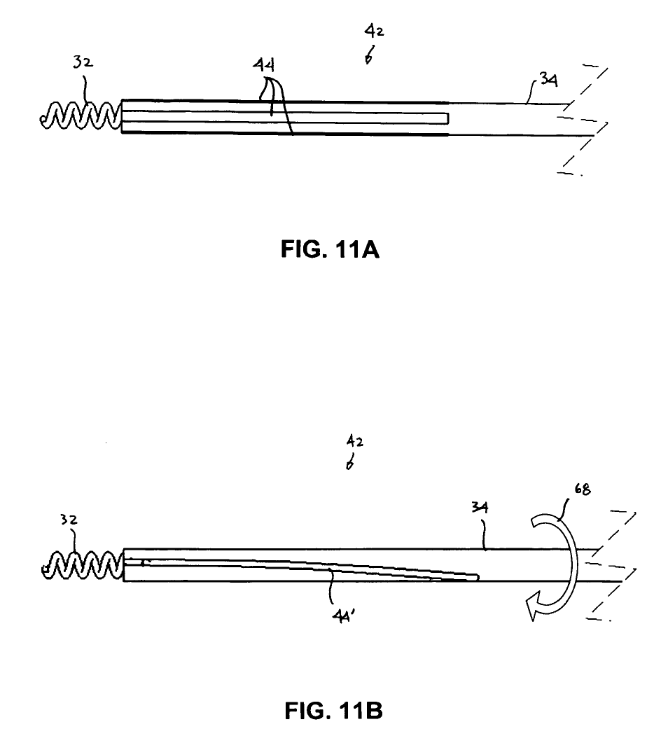

As described above, marked section 42 may be utilized as a visual indicator for determining the direction of shaft rotation when advancing or withdrawing tissue engagement member 32 into tissue. As shown in FIGS. 11A and 11B, the longitudinal stripes 44, in particular, may be utilized to indicate a state of over-torquing shaft 34 against tissue. FIG. 11A shows shaft 34 with longitudinal stripes 44 in a straight configuration, which is indicative of a relatively unstressed shaft when piercing tissue with engagement member 32. Upon fully inserting engagement member 32 into the tissue region, the tissue surface may begin to wind or wad around the shaft 34. As shaft 34 is further torqued, as indicated by the direction of torquing 68 in FIG. 11B, longitudinal stripe 44 may begin to take a curved shape 44′ visually indicating to the user that the shaft 34 is being over-torqued relative to the engaged tissue region.

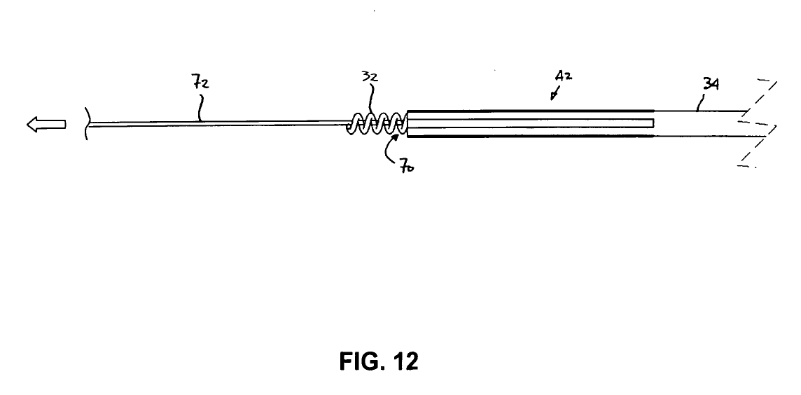

In addition to the marked section 42 for visually facilitating tissue engagement and manipulation, flexible shaft 34 may also define a hollow lumen 70 extending through the length of shaft 34 and terminating in an opening defined at a distal tip of shaft 34. The hollow lumen 70 may be sized appropriately for a guidewire 72 to be passed therethrough distally through the opening and through the center of helical tissue engagement member 32, as shown in FIG. 12. Guidewire 72 may be passed through shaft 34 and through helical tissue engagement member 32 to be utilized as a guide during intra-luminal or trans-luminal procedures. For example, when utilizing flexible shaft 34 in a transgastric procedure for passing an endoluminally advanced shaft 34 from within the stomach and into the peritoneal cavity, guidewire 72 may be initially advanced through a gastrotomy with shaft 34 and engagement member 32 being advanced over the transgastrically positioned guidewire 72 and into the peritoneal cavity of a patient body.

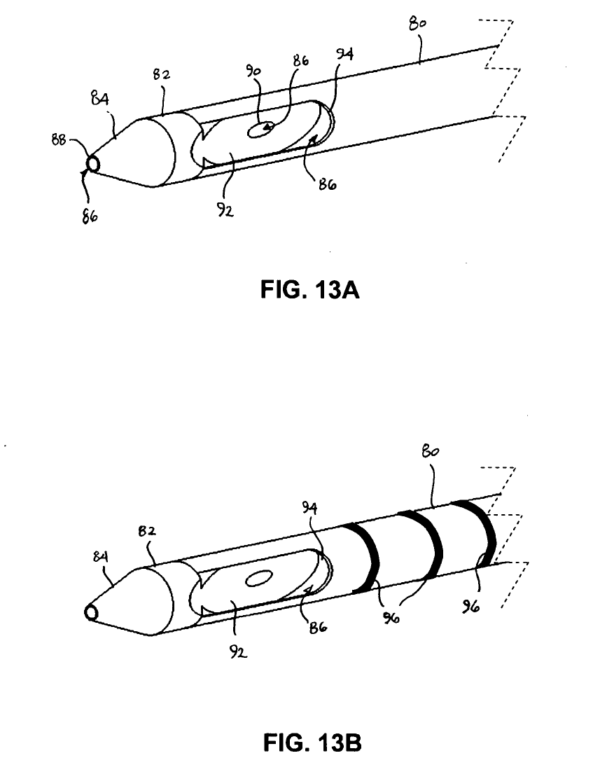

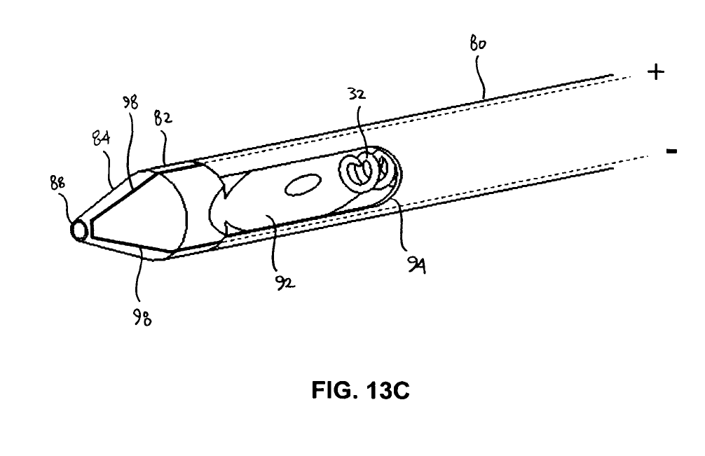

FIG. 13A shows a perspective view of an optional dilating sheath 80 which may be particularly useful for facilitating transgastric entry of the flexible shaft 34 through a gastrotomy from within the stomach and into the peritoneal cavity. Dilating sheath 80 may be advanced endoluminally through a lumen 28 within the elongate body 12 and sheath 80 may generally comprise a flexible tubular member with a tip portion 82 attached to a distal end of the sheath 80. Tip portion 82 may be attached as a separate tip or may be formed as an integral part of sheath 80 and may define a dilating or tapered tip 84 for facilitating dilation of a tissue opening. Tip portion 82 may define a guidewire lumen 86 through the tip 82 with a distal tip opening 88 at the end of tip 82 for passage of the guidewire 72 therethrough. Guidewire lumen 86 may pass through the tip portion 82 to proximal tip opening 90. A proximal portion of tip 82 may further define a ramp or angled surface 92 adjacent to an opening or skive 94 defined in the distal end of sheath 80.

FIG. 13B shows another variation of sheath 80 having one or more visual indicators 96 defined over the outer surface of sheath 80. Visual indicators 96 may be defined as circumferential markings, as shown, in the figure or any other markings, designs, patterns, projections, textures, etc., and in various colors, as described above, which acts to provide a visual indication to the user as to the translational movement, rotation, direction of rotation, etc., of sheath 80.

FIG. 13C shows yet another variation of sheath 80 having an optional energizable wire, e.g., made from Nickel-Chromium, which may be integrated with the sheath 80 for facilitating tissue dilation and/or cutting. As shown, wire 98 may be connected to a power supply at its proximal end located external to the patient body and the wire 98 may be routed along an outer surface of sheath 80 or through a lumen defined through sheath 80. Around or upon tip 82, wire 98 may be positioned along or around tapered tip 84. When tip 82 is advanced through, e.g., a gastrotomy, which requires widening, wire 98 may be energized to cut any contacted tissue to facilitate widening the opening.

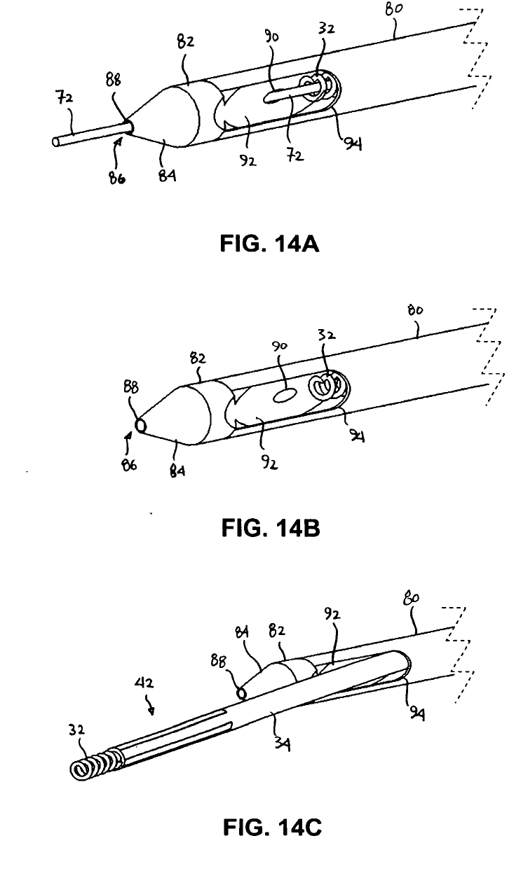

FIGS. 14A to 14C show perspective views of one example for using a tissue engagement instrument and a guidewire with the optional dilating sheath 80, which may be particularly useful for facilitating transgastric entry of the flexible shaft 34 through a gastrotomy from within the stomach and into the peritoneal cavity. Examples for utilizing a helical engagement member 32 and flexible shaft 34 in transgastric procedures, which may be utilized with the instruments and methods described herein, are described in further detail in U.S. Prov. Pat. App. Serial No. 60/728,382 filed Oct. 18, 2005, which is incorporated herein by reference in its entirety. For example, with elongate body 12 positioned trans-esophageally within the stomach of a patient body, a guidewire 72 may be advanced through a lumen, e.g., lumen 28, of the elongate body 12. Upon the formation of a gastrotomy from within the stomach, guidewire 72 may be passed through the gastrotomy and into the peritoneal cavity. To accommodate the passage of guidewire 72 within sheath 80, the guidewire 72 may be passed, as described above, through helical engagement member 32 and flexible shaft 34 positioned within sheath 80 and through the guidewire lumen 86 defined through tip portion 82, as shown in FIG. 14A. When guidewire 72 is positioned through both helical engagement member 32 and through guidewire lumen 86, the guidewire 72 functions additionally as a locking mechanism which prevents or inhibits the inadvertent pushing of helical engagement member 32 and shaft 34 outside the opening or skive 94 of sheath 80.

Once the guidewire 72 has been advanced through the gastrotomy, sheath 80 may be advanced over guidewire 72 through the gastrotomy and into the peritoneal cavity while dilating the opening via tapered tip 84. Once the distal portion of sheath 80, or at least opening or skive 94, has been advanced into the peritoneal cavity, guidewire 72 may be withdrawn (entirely or partially) from sheath 80 until at least guidewire 72 has been withdrawn from tip portion 82 and helical engagement member 32, as shown in FIG. 14B. Once free, flexible shaft 34 may be urged distally through sheath 80 such that helical engagement member 32 is advanced out of opening 94 by ramp or angled surface 92, as shown in FIG. 14C. Helical engagement member 32 may be then advanced distally further into the peritoneal cavity or into proximity against a tissue region to be manipulated by the engagement member 32. Optionally, guidewire 72 may be re-advanced distally through flexible shaft 34 to re-emerge from engagement member 32 for use within the peritoneal cavity.

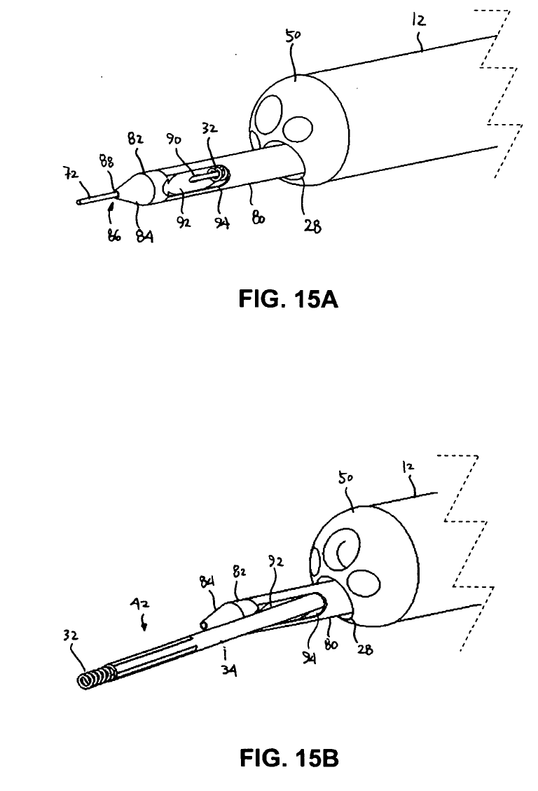

FIGS. 15A and 15B show detail perspective views of an example showing sheath 80 positioned within lumen 28 of elongate body 12. As described above, sheath 80 may be advanced distally through lumen 28 over or along guidewire 72 with helical engagement member 32 and flexible shaft 34 positioned within sheath 80, as shown in FIG. 15A. Once the guidewire 72, for instance, has been advanced transgastrically, sheath 80 may be advanced over guidewire 72 in a low-profile configuration past the gastrotomy and guidewire 72 may be withdrawn proximally until engagement member 32 is free to be advanced along ramp 92 and out of opening 94 and into the peritoneal cavity or other region within the body, as shown in FIG. 15B.

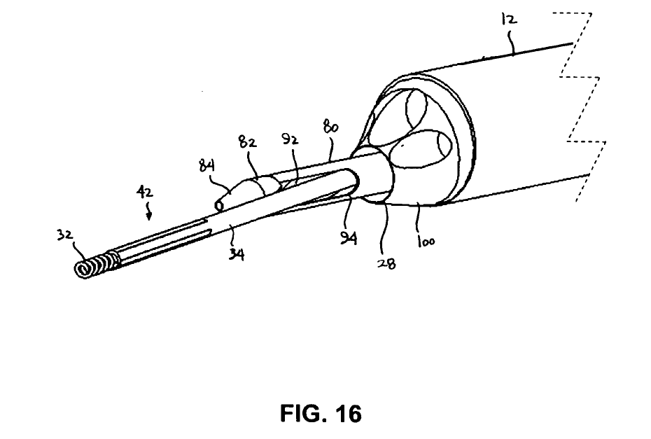

FIG. 16 shows yet another variation with sheath 80 and flexible shaft 34 positioned through opening 94. In this variation, the distal end of elongate body 12 may include a tapered atraumatic tip 100 which is eccentrically angled such that the opening of lumen 28, where sheath 80 and flexible shaft 34 exit, defines the distal apex of the tapered tip 100. This eccentrically angled surface of tip 100 may be utilized as a dilating surface for widening the tissue opening, e.g., gastrotomy, and facilitating the passage of elongate body 12 through the gastrotomy when sheath 80 and/or engagement member 32 and flexible shaft 34 have already been passed through the gastrotomy.

Although a number of illustrative variations are described above, it will be apparent to those skilled in the art that various changes and modifications may be made thereto without departing from the scope of the invention. Moreover, although specific configurations and applications may be shown, it is intended that the various features may be utilized in various combinations and in various types of procedures as practicable. It is intended in the appended claims to cover all such changes and modifications that fall within the true spirit and scope of the invention.

What is claimed is:

1. A tissue manipulation instrument, comprising:
an elongate flexible shaft having a proximal end and a distal end; and
a helical tissue engager adapted to reversibly engage tissue positioned upon the distal end of the flexible shaft,
wherein at least one portion of an outer surface of the flexible shaft defines a marked section adapted to provide a visual indication of movement of the flexible shaft and the helical tissue engager.

2. The instrument of claim 1 wherein the helical tissue engager comprises a tissue piercing helix or corkscrew structure.

3. The instrument of claim 1 wherein the helical tissue engager further comprises an elongated length extending between the helical tissue engager and the distal end of the flexible shaft.

4. The instrument of claim 1 wherein the marked section is defined on the outer surface of the flexible shaft proximal to the helical tissue engager.

5. The instrument of claim 4 wherein the marked section is defined along at least a majority of the length of the flexible shaft.

6. The instrument of claim 1 wherein the marked section is comprised of markings, designs, patterns, projections, or textures to provide the visual indication of movement.

7. The instrument of claim 1 wherein the marked section is adapted to provide the visual indication with respect to translational movement, rotation, or direction of rotation of the flexible shaft and the helical tissue engager with respect to a tissue surface.

8. The instrument of claim 1 wherein the marked section is comprised of a plurality of longitudinal stripes defined along a length of the flexible shaft.

9. The instrument of claim 1 further comprising a handle on the proximal end of the flexible shaft.

10. The instrument of claim 9 wherein the handle is removable.

11. The instrument of claim 1 further comprising a power supply connectable to the flexible shaft and in electrical communication with at least a portion of the helical tissue engager.

12. The instrument of claim 1 wherein the flexible shaft further comprises a band having a predetermined length defined upon the distal end of the flexible shaft proximal to the helical tissue engager.

13. The instrument of claim 1 wherein the flexible shaft further comprises a circumferential stop positioned at the distal end of the flexible shaft.

14. The instrument of claim 1 wherein the marked section comprises a spiral pattern.

15. The instrument of claim 1 further comprising a guidewire positionable through a hollow lumen defined through the flexible shaft and through the helical tissue engager.

16. The instrument of claim 1 further comprising a dilating sheath, comprising:
a flexible tubular member defining a lumen for passage of the helical tissue engager therethrough;
a tip portion attached to a distal end of the tubular member;
wherein the tip portion defines a tapered tip at a distal end and a ramped surface at a proximal end with a lumen defined therethrough, and
wherein the flexible tubular member defines an opening adjacent to the ramped surface for passage of the helical tissue engager therethrough.

17. The instrument of claim 16 wherein an outer surface of the tubular member further defines visual indicators thereon.

18. The instrument of claim 16 wherein the tip portion is energizable for cutting tissue.

19. The instrument of claim 1 further comprising a tissue grasping tool pivotably coupled to a distal end of an elongate member and adapted to be advanced endoluminally into a body lumen.

20. The instrument of claim 19 wherein the tissue grasping tool comprises a first jaw member pivotably coupled to the distal end of the elongate member, a second jaw member pivotably coupled along the first jaw member, and a launch tube member adapted to urge the first and second jaw members between a low-profile delivery configuration and an expanded grasping configuration.

21. The instrument of claim 1 further comprising an endoscopic device through which the tissue manipulation instrument is advanceable through, the endoscopic device having a flexible body with a steerable distal section, wherein the flexible body is adapted to be rigidized to maintain an arbitrary shape.

22. A method of endoluminally manipulating a region of tissue, comprising:
endoluminally advancing a helical tissue engager positioned upon a distal end of an elongate flexible shaft, the tissue engager being adapted to reversibly engage tissue;
rotating the flexible shaft such that the region of tissue is engaged by the helical tissue engager; and
viewing a marked section on an outer surface of the flexible shaft to indicate movement of the flexible shaft and the helical tissue engager while rotating the flexible shaft.

23. The method of claim 22 wherein endoluminally advancing comprises advancing the helical tissue engager transesophageally into a stomach.

24. The method of claim 22 wherein endoluminally advancing comprises advancing an endoscopic device having a flexible body with a steerable distal section trans-esophageally, wherein the flexible body is adapted to be rigidized to maintain an arbitrary shape.

25. The method of claim 22 wherein rotating the flexible shaft comprises torquing a handle at a proximal end of the flexible shaft.

26. The method of claim 22 wherein viewing a marked section comprises viewing a rotation of at least one longitudinal stripe defined along the outer surface of the flexible shaft.

27. The method of claim 26 wherein viewing a rotation comprises watching for the at least one longitudinal stripe to conform into a curved shape to visually indicate an over-torqued condition of the flexible shaft relative to the engaged region of tissue.

28. The method of claim 22 further comprising energizing the helical tissue engager while rotating the flexible shaft to engage the region of tissue.

29. The method of claim 22 further comprising advancing a guidewire through a lumen defined through the flexible shaft and through the helical tissue engager while endoluminally advancing a helical tissue engager.

30. The method of claim 29 further comprising advancing the guidewire and the flexible shaft through a flexible tubular sheath having a tapered tip and a ramped surface adapted to direct the helical tissue engager through an opening defined along the tubular sheath.

31. The method of claim 30 further comprising energizing a portion of the tapered tip of the flexible tubular sheath.
